(19) United States
(12) Patent Application Publication
(10) Pub. No.: US 2013/0268587 A1
(43) Pub. Date: Oct. 10, 2013

(54) INFORMATION PROCESSING DEVICE AND DATA MANAGEMENT SYSTEM

(71) Applicant: Yutaka Ueda, Hachioji-shi (JP)

(72) Inventor: Yutaka Ueda, Hachioji-shi (JP)

(73) Assignee: KONICA MINOLTA, INC., Chiyoda-ku, Tokyo (JP)

(21) Appl. No.: 13/994,408

(22) PCT Filed: Dec. 1, 2011
(86) PCT No.: PCT/JP2011/077746

§ 371 Date: Jun. 14, 2013

(30) Foreign Application Priority Data
Dec. 14, 2010 (JP) 2010-277582

(51) Int. Cl. H04L 29/06

(52) U.S. Cl. CPC H04L 67/42
USPC 709/203

(57) ABSTRACT

An information processing device includes a first storage unit, a transmission unit, a delete unit and a display control unit. The transmission unit transmits image data acquired from an examination device and stored in the first storage unit to a data management server to store the image data in a second storage unit of the data management server. The delete unit deletes the image data from the first storage unit if a predetermined condition is satisfied. The display control unit searches the first and second storage units for the image data, acquires the image data from the first storage unit with respect to the image data stored in the first storage unit, and acquires the image data from the second storage unit with respect to the image data not stored in the first storage unit.

### TECHNICAL FIELD OF THE INVENTION

The present invention relates to an information processing device and a data management system.

### BACKGROUND OF THE ART

In recent years, in the medical field, there has been used a system to store medical-use images taken with radiation or the like as digital data. Usually, medical-use images are stored in an image management server installed in a hospital, and image data thereof in the image management server is loaded into client terminals so that the medical-use images are displayed on the client terminals.

However, in the case where an image management server is installed in a hospital, an expensive high-spec PC (Personal Computer) is necessary to operate as a server, and hence installation of an image management server costs much. Furthermore, in order that data in a hospital is looked at from the outside, it is necessary to make the image management server keep operating because images cannot be looked at when power of the image management server is off.

Then, to reduce the cost of storing and managing data to be paid by medical facilities, there has been proposed a medical data storage system to transmit medical data, such as medical-use image data taken at medical facilities, to a data center connected to the medical facilities via a network so as to accumulate and manage the medical data at the data center. (Refer to Patent Document 1 and Patent Document 2).

### RELATED ART DOCUMENT

### Patent Document

Patent Document 1: Japanese Patent Application Laid-Open Publication No. 2005-267549

Patent Document 2: Japanese Patent Application Laid-Open Publication No. 2005-267551

### SUMMARY OF THE INVENTION

### The Problems to be Solved by the Invention

However, if medical-use image data is stored in an image management server connected to medical facilities with not-high-speed communication line, it takes time to display images, and hence diagnostic efficiency decreases.

The present invention is made in view of the above-described problems of the conventional technology, and an object of the present invention is to prevent the diagnostic efficiency from decreasing.

### Means for Solving the Problems

In order to solve the above-described problems, the invention described in claim 1 is an information processing device including: an acquirement unit which acquires image data from an examination device; a first storage unit which temporarily stores the image data acquired from the examination device; a transmission unit which transmits the image data stored in the first storage unit to a data management server so as to store the image data in a second storage unit of the data management server connected to the transmission unit via a communication network so as to perform data communication; a delete unit which deletes the image data stored in the first storage unit from the first storage unit in response to a predetermined condition being satisfied; and a display control unit which searches the first storage unit and the second storage unit for the image data, acquires the image data from the first storage unit and displays an image on a display unit on the basis of the image data acquired from the first storage unit with respect to the image data stored in the first storage unit, and acquires the image data from the second storage unit and displays an image on the display unit on the basis of the image data acquired from the second storage unit with respect to the image data not stored in the first storage unit.

The invention described in claim 2 is the information processing device according to claim 1, wherein the delete unit deletes the image data stored in the first storage unit from the first storage unit in response to a predetermined period elapsing from when the image data is stored in the first storage unit.

The invention described in claim 3 is the information processing device according to claim 1 or 2, wherein a process performed by the information processing device is performed in accordance with an application program downloaded from the data management server, and the information processing device further comprises a program update unit which, in response to a newer application program than the downloaded application program existing in the data management server, downloads the newer application program from the data management server.

The invention described in claim 4 is a data management system including: an information processing device including a first storage unit which temporarily stores image data acquired from an examination device; and a data management server including a second storage unit which stores the image data transmitted from the information processing device connected to the data management server via a communication network so as to perform data communication, wherein the information processing device includes: a transmission unit which transmits the image data stored in the first storage unit to the data management server so as to store the image data in the second storage unit; a delete unit which deletes the image data stored in the first storage unit from the first storage unit in response to a predetermined condition being satisfied; and a display control unit which searches the first storage unit and the second storage unit for the image data, acquires the image data from the first storage unit and displays an image on a display unit on the basis of the image data stored in the first storage unit with respect to the image data stored in the first storage unit, and acquires the image data from the second storage unit and displays an image on the display unit on the basis of the image data stored in the second storage unit with respect to the image data not stored in the first storage unit.

The invention described in claim 5 is the data management system according to claim 4, wherein the delete unit deletes the image data stored in the first storage unit from the first storage unit in response to a predetermined period elapsing from when the image data is stored in the first storage unit.

The invention described in claim 6 is the data management system according to claim 4 or 5, wherein a process performed by the information processing device is performed in accordance with an application program downloaded from the data management server, and the information processing device further includes a program update unit which, in response to a newer application program than the downloaded application program existing in the data management server, downloads the newer application program from the data management server.

The invention described in claim 7 is the data management system according to claim 6, wherein the data management server further includes a fee calculation unit which calculates a fee for use of the data management system by the information processing device on the basis of a data amount of the image data stored in the second storage unit and a number of times the application program is used.

### Effects of the Invention

According to the inventions described in claims 1, 2, 4 and 5, the image data stored in the first storage unit is acquired from the first storage unit, and the image data not stored in the first storage unit is acquired from the second storage unit. Accordingly, time necessary for displaying images can be reduced, and the diagnostic efficiency can be prevented from decreasing.

According to the inventions described in claims 3 and 6, the latest application program can be used always.

According to the present invention described in claim 7, fees can be calculated in accordance with the use of the data management system by the information processing device.

### BRIEF DESCRIPTION OF THE DRAWINGS

FIG. 1 is a system configuration diagram of a data management system.

FIG. 2 is a block diagram showing the functional configuration of the data management server.

FIG. 3 is an example of a facility information table contained in a center DB.

FIG. 4 is an example of an image information table contained in the center DB.

FIG. 5 is a block diagram showing the functional configuration of a terminal device included in a medical facility A.

FIG. 6 is an example of a facility information table contained in a primary DB.

FIG. 7 is an example of an image information table contained in the primary DB.

FIG. 8 is a flowchart showing an application start time process performed by the terminal device.

FIG. 9 is a flowchart showing an image capture process performed by the terminal device.

FIG. 10 is an example of a received patient list screen.

FIG. 11 is an example of a viewer screen.

FIG. 12 is a flowchart showing an image display process performed by the terminal device.

FIG. 13 is an example of a viewer screen.

FIG. 14 is a flowchart showing a fee calculation process performed by the data management server.

### MODE FOR CARRYING OUT THE INVENTION

In the following, an embodiment of a data management system of the present invention is described with reference to the drawings. However, the scope of the present invention is not limited to the illustrated embodiment.

### [Configuration of Data Management System]

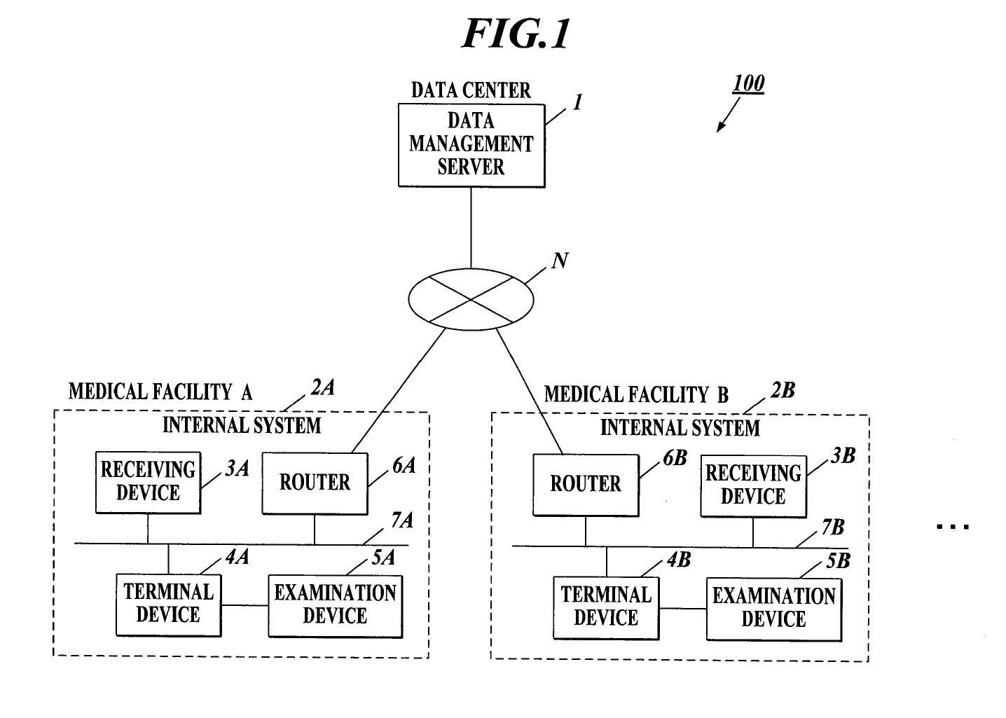

FIG. 1 shows the system configuration of a data management system 100 of the embodiment.

As shown in FIG. 1, the data management system 100 includes a data management server 1 installed in a data center in a region and internal systems 2A, 2B and . . . respectively disposed in medical facilities A, B and . . . . The data management server 1 and the internal systems 2A, 2B and . . . are connected to each other via a communication network N, such as Internet, so as to communicate with each other.

The data management server 1 accumulates and manages image data of medical-use images generated at the medical facilities. The data management server 1 provides medical cooperative service among the medical facilities, and receives, from an internal system of the internal systems 2A, 2B and . . . , requests to other internal systems thereof for examinations or radiographic image interpretations, and manages data of the results of the examinations or radiographic image interpretations.

The internal system 2A includes a receiving device 3A, a terminal device 4A, an examination device 5A and a router 6A. The receiving device 3A, the terminal device 4A and the router 6A are connected to each other via an internal network 7A, such as a LAN (Local Area Network), so as to perform data communication. Of the devices constituting the internal system 2A, at least the terminal device 4A is connected to the data management server 1 via the communication network N with the router 6A so as to perform data communication.

The internal system 2B includes a receiving device 3B, a terminal device 4B, an examination device 5B and a router 6B. The receiving device 3B, the terminal device 4B and the router 6B are connected to each other via an internal network 7B, such as a LAN, so as to perform data communication. Of the devices constituting the internal system 2B, at least the terminal device 4B is connected to the data management server 1 via the communication network N with the router 6B so as to perform data communication.

The number of internal systems such as the internal systems 2A and 2B which constitute the data management system 100 and the number of devices which constitute each internal system are not particularly limited.

### [Configuration of Data Management Server]

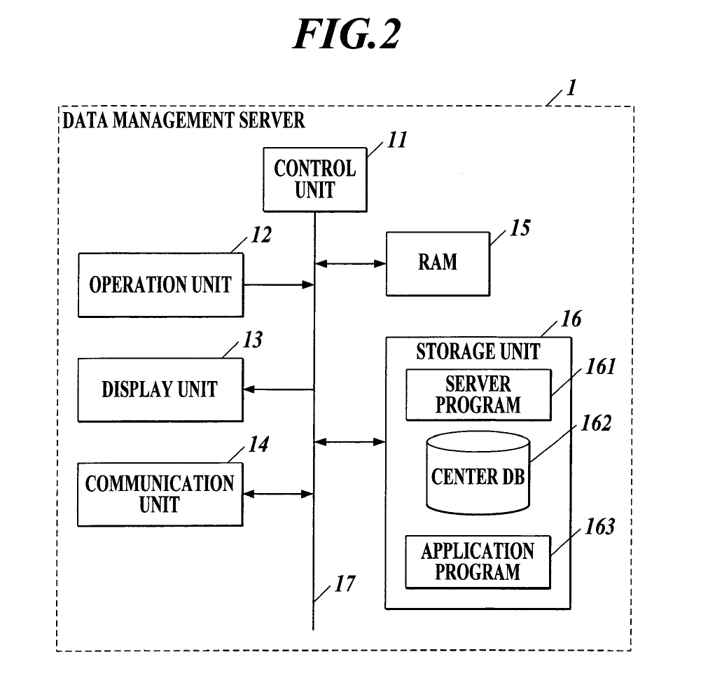

FIG. 2 shows the functional configuration of the data management server 1.

As shown in FIG. 2, the data management server 1 includes a control unit 11, an operation unit 12, a display unit 13, a communication unit 14, a RAM 15 and a storage unit 16, and these components are connected to each other via a bus 17.

The control unit 11 is constituted of a CPU (Central Process Unit) or the like, and controls process operations of the components of the data management server 1 comprehensively. More specifically, the CPU reads various process programs stored in the storage unit 16 so as to open the read programs in the RAM 15 in response to operation signals inputted from the operation unit 12 or command signals received by the communication unit 14 so as to perform various processes in cooperation with the programs.

The operation unit 12 includes a keyboard including cursor keys, number input keys and various function keys, and a pointing device such as a mouse, and outputs operation signals inputted by key operations on the keyboard or mouse operations to the control unit 11.

The display unit 13 includes a monitor such as an LCD (Liquid Crystal Display), and displays various screens thereon in response to commands of display signals inputted from the control unit 11.

The communication unit 14 is constituted of a network interface or the like, and transmits/receives data to/from an external device connected to the communication unit 14 via the communication network N.

The RAM 15 forms a work area to temporarily store various programs, input/output data, parameters and the like read from the storage unit 16 in various processes executed and controlled by the control unit 11.

The storage unit 16 is constituted of an HDD (Hard Disk Drive), a nonvolatile semiconductor memory or the like, and stores various process programs, and parameters, files and the like necessary for execution of the programs. More specifically, the storage unit 16 stores a server program 161, a center DB (DataBase) 162, an application program 163 and the like.

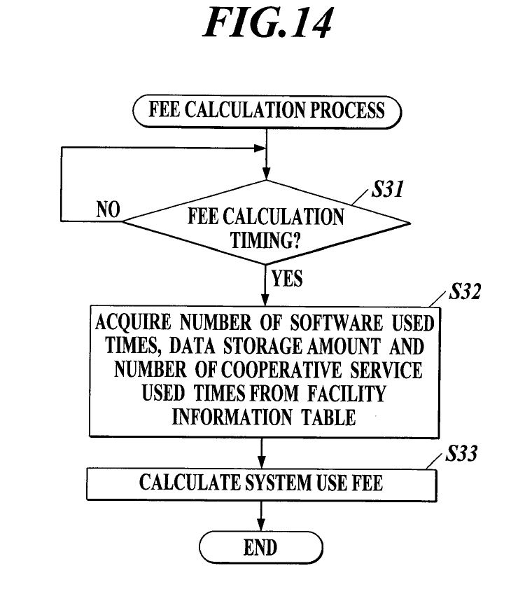

The server program 161 is a program to perform a data management process, a process to provide the application program 163, a process to provide the medical cooperative service, a fee calculation process (see FIG. 14) and the like in the data management server 1.

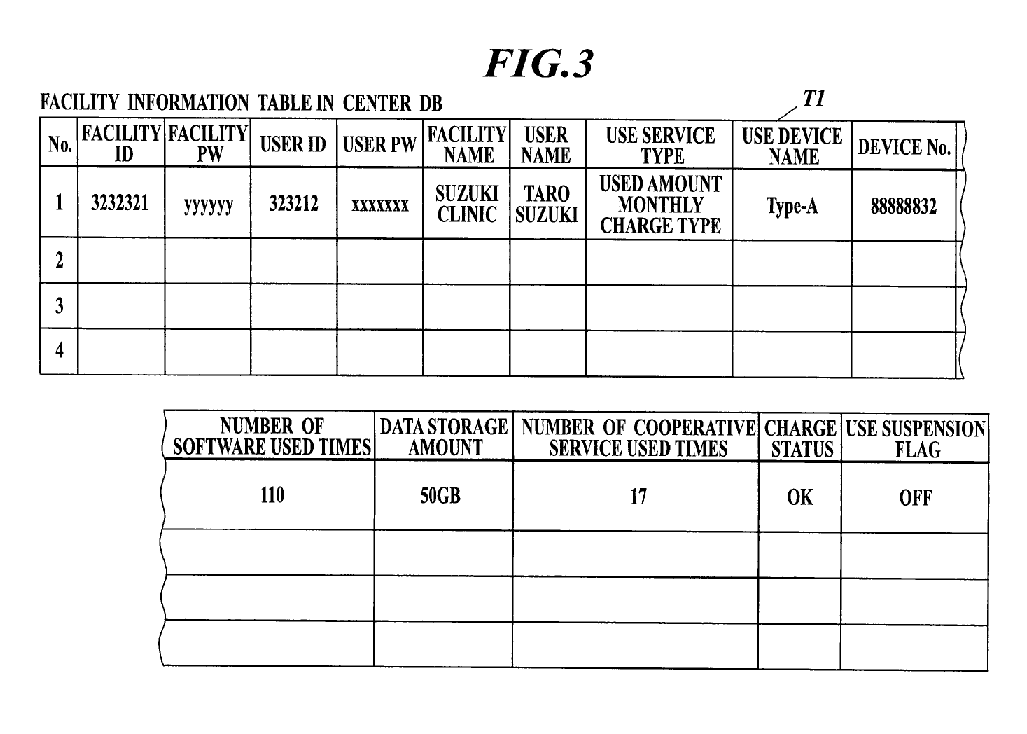

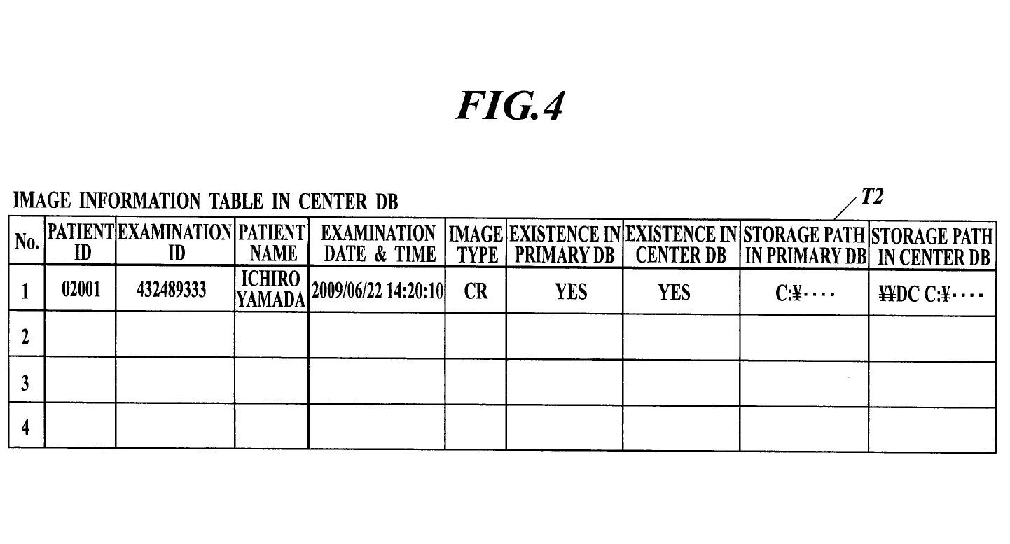

The center DB 162 contains a facility information table T1 (see FIG. 3) and an image information table T2 (see FIG. 4). The center DB 162 stores image data transmitted from the terminal devices 4A, 4B and . . . of the medical facilities A, B and . . . .

FIG. 3 is an example of the facility information table T1 contained in the center DB 162. The facility information table T1 stores information on the medical facilities included in the data management system 100. More specifically, the facility information table T1 stores a facility ID, a facility PW (Password), a user ID, a user PW, a facility name, a user name, a use service type, a use device name, a device No., a number of software used times, a data storage amount, a number of cooperative service used times, a charge status and a use suspension flag with respect to each medical facility.

The facility ID and the facility PW are identification information and a password, respectively, allotted to each medical facility.

The user ID and the user PW are identification information and a password, respectively, allotted to each user.

The use service type is information on a type of charge for use of the data management system 100.

The use device name and the device No. are a machine name and a machine number, respectively, of the examination device 5A, 5B or . . . used at each medical facility.

The number of software used times is the number of times (for a month, in total or the like) software (an application program 461 described below) is used by each medical facility.

The data storage amount is a data amount (for a month, in total or the like) stored in the center DB 162 of the storage unit 16 with respect to each medical facility.

The number of cooperative service used times is the number of times (for a month, in total or the like) the cooperative service (requests to other medical facilities for examinations, radiographic image interpretations or the like) is used by each medical facility.

The charge status is information on whether or not a fee is paid by each medical facility. The charge status can be acquired from another system (a sales/distribution system or the like) of the service provider.

The use suspension flag is a flag to specify whether or not to stop each medical facility from using the data management system 100.

FIG. 4 is an example of the image information table T2 contained in the center DB 162. The image information table T2 is prepared for each medical facility. The image information table T2 stores information on images belonging to examinations performed at a medical facility. More specifically, the image information table T2 stores a patient ID, an examination ID, a patient name, an examination date & time, an image type, existence in primary DB, existence in center DB, a storage path in primary DB and a storage path in center DB.

The patient ID is identification information on a patient of a medical facility.

The examination ID is identification information on an examination.

The patient name is the name of a patient.

The examination date & time is a date and time of an examination performed.

The image type is information on a type of an image. Examples of the image type include CR (Computed Radiography), CT (Computed Tomography) and MRI (Magnetic Resonance Imaging).

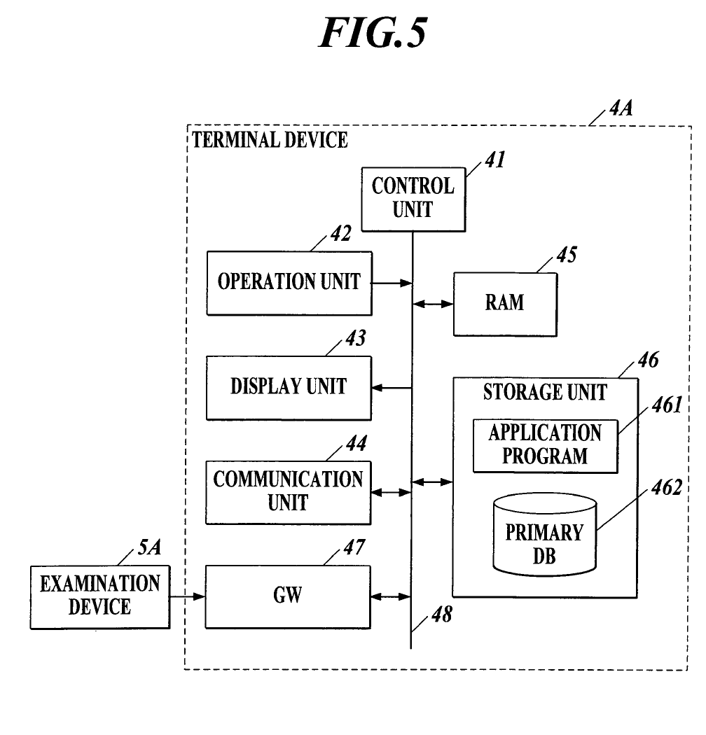

The existence in primary DE is information on whether or not image data generated by an examination is stored in a primary DB 462 (see FIG. 5) of the medical facility.

The existence in center DB is information on whether or not image data generated by an examination is stored in the center DB 162.

The storage path in primary DB is information on a storage location of image data in a case where image data generated by an examination is stored in the primary DB 462 (see FIG. 5) of the medical facility.

The storage path in center DB is information on a storage location of image data in a case where image data generated by an examination is stored in the center DE 162.

The application program 163 is a program to be downloaded by the terminal devices 4A, 4B and . . . of the internal systems 2A, 2B and . . . , and is replaced with its newer program whenever upgraded. The application program 163 is a program to make the terminal devices 4A, 4B and . . . realize a list display function, a viewer function, an image process function such as enlargement, reduction or rotation, a measurement function, an output function to output images to films, paper or media, and the like.

The control unit 11 receives image data and accessory information (the patient ID, the examination ID, the patient name, the examination date & time, the image type, the storage path in primary DB and the like) on the image data from the terminal device 4A, 4B or . . . with the communication unit 14. Then, the control unit 11 stores the received image data in the center DB 162 of the storage unit 16, and adds a new record to the image information table T2 on the basis of the received accessory information (the patient ID, the examination ID, the patient name, the examination date & time, the image type, the storage path in primary DB and the like). In addition, with respect to the corresponding examination in the image information table T2, the control unit 11 puts “YES” in the “existence in primary DB”, puts “YES” in the “existence in center DB”, and adds a storage location of the image data in the center DB 162 to the “storage path in center DB”. Then, the control unit 11 controls the communication unit 14 so that a notice that the image data is stored in the center DB 162 and the storage location of the image data in the center. DE 162 are transmitted to the terminal device 4A, 4B or . . . which has transmitted the image data to the control unit 11. As the image data is stored in the center DB 162, the control unit 11 updates, on the basis of the data amount of the newly stored image data, the “data storage amount” in the facility information table T1 with respect to the medical facility which has performed the examination.

When the control unit 11 receives a notice that image data is deleted from the primary DB 462 (see FIG. 5) from the terminal device 4A, 4B or . . . of a medical facility with the communication unit 14, with respect to the corresponding examination in the image information table T2 with respect to the medical facility, the control unit 11 changes the “existence in primary DB” to “NO”, and deletes the storage location of the image data in the primary DB 462 from the “storage path in primary DB”.

When the control unit 11 receives an acquirement request for the application program 163 from the terminal device 4A, 4B or . . . of a medical facility with the communication unit 14, the control unit 11 controls the communication unit 14 so that the application program 163 stored in the storage unit 16 is transmitted to the terminal devices 4A, 4B or . . . which has transmitted the request.

The control unit 11 receives, from a medical facility included in the data management system 100, a request for an examination, radiographic image interpretation or the like to another medical facility included therein, and notifies the another medical facility of the request for the examination, radiographic image interpretation or the like. In addition, the control unit 11 acquires, from the another medical facility, examination data of the examination performed in response to the request for the examination, report data of the radiographic image interpretation performed in response to the request for the radiographic image interpretation, or the like, and provides it to the medical facility which has transmitted the request.

When software (the application program 461 described below) is used by the terminal device 4A, 4B or . . . of a medical facility, the control unit 11 acquires information on the use of the software from the terminal device 4A, 4B or . . . which has used the software, and updates the “number of software used times” in the facility information table T1 with respect to the medical facility. In addition, when the cooperative service is used, namely, for example, when the control unit 11 receives, from the terminal 4A, 4B or . . . of a medical facility, a request for an examination, radiographic image interpretation or the like to another medical facility, the control unit 11 updates the “number of cooperative service used times” in the facility information table T1 with respect to the medical facility.

The control unit 11 calculates fees for use of the data management system 100 with respect to the terminal devices 4A, 4B and . . . on the basis of the facility information table T1. That is, the control unit 11 functions as a fee calculation unit. More specifically, the control unit 11 charges each medical facility a variable fee in accordance with the “number of software used times”, the “data storage amount” and the “number of cooperative service used times”.

Alternatively, it is possible that a fixed fee is set with the “number of software used times” unlimited, and a variable fee is charged if the “data storage amount” and the “number of cooperative service used times” exceed their respective predetermined upper limits.

### [Configuration of Internal System]

Next, the devices constituting the internal system 2A are described.

The receiving device 3A is a computer device including a control unit, an operation unit, a display unit, a communication unit, a RAM and a storage unit, and performs a reception process, an accounting calculation and an insurance point calculation for patients who come to the medical facility A. When a patient comes to the medical facility A, in the receiving device 3A, a patient name and a patient ID (identification information on the patient at the medical facility A) are inputted from the operation unit, a receipt number is allotted to the patient in order of reception with the earliest one first, and the receipt number and the patient name are correlated with each other and stored in the storage unit.

The terminal device 4A is an information processing device used for capturing image data from the examination device 5A, referring to an image generated by an examination, making a request for an examination or radiographic image interpretation to be performed at another medical facility to the data management system 1, or the like.

FIG. 5 shows the functional configuration of the terminal device 4A.

As shown in FIG. 5, the terminal device 4A includes a control unit 41, an operation unit 42, a display unit 43, a communication unit 44, a RAM 45, a storage unit 46 and a GW (GateWay) 47, and these components are connected to each other via a bus 48.

The control unit 41 is constituted of a CPU (Central Process Unit) or the like, and controls process operations of the components of the terminal device 4A comprehensively. More specifically, the CPU reads various process programs stored in the storage unit 46 so as to open the read programs in the RAM 45 in response to operation signals inputted from the operation unit 42 or command signals received by the communication unit 44 so as to perform various processes in cooperation with the programs.

The operation unit 42 includes a keyboard including cursor keys, number input keys and various function keys, and a pointing device such as a mouse, and outputs operation signals inputted by key operations on the keyboard or mouse operations to the control unit 41.

The display unit 43 includes a monitor such as an LCD, and displays various screens thereon in response to commands of display signals inputted from the control unit 41.

The communication unit 44 is constituted of a network interface or the like, and transmits/receives data to/from an external device connected to the communication unit 44 via the internal network 7A and the communication network N.

The RAM 45 forms a work area to temporarily store various programs, input/output data, parameters and the like read from the storage unit 46 in various processes executed and controlled by the control unit 41.

The storage unit 46 is constituted of an HDD, a nonvolatile semiconductor memory or the like, and stores various process programs, and parameters, files and the like necessary for execution of the programs. More specifically, the storage unit 46 stores the application program 461, the primary DB 462 and the like.

The application program 461 is a program (the application program 163) to perform a process to use the data management system 100 in the terminal device 4A, and is downloaded from the data management server 1.

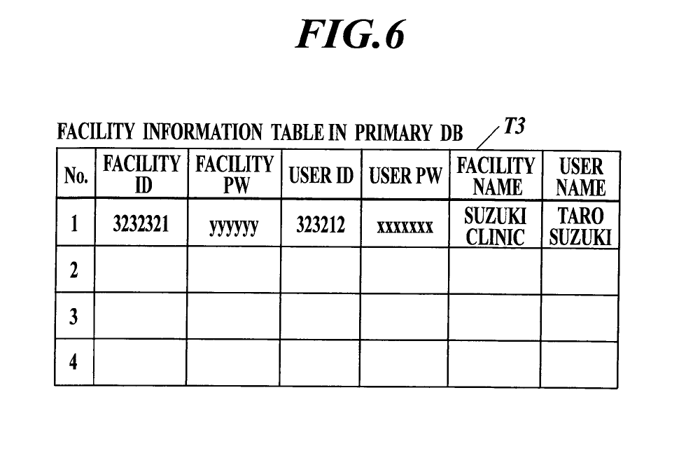

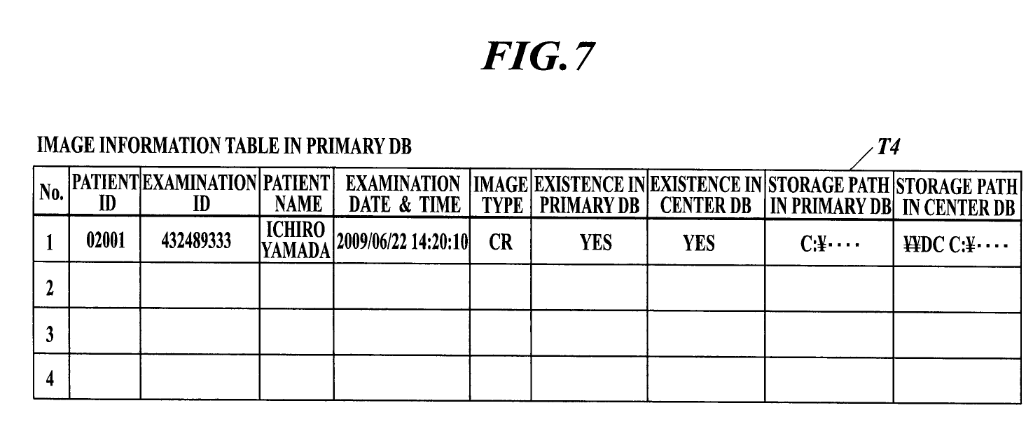

The primary DB 462 contains a facility information table T3 (see FIG. 6) and an image information table T4 (see FIG. 7). The primary DB 462 temporarily stores image data acquired from the examination device 5A.

FIG. 6 is an example of the facility information table T3 contained in the primary DB 462. The facility information table T3 stores information on the medical facility A. More specifically, the facility information table T3 stores a facility ID, a facility PW, a user ID, a user PW, a facility name and a user name.

FIG. 7 is an example of the image information table T4 contained in the primary DB 462. The image information table T4 stores information on images belonging to examinations performed at the medical facility A. More specifically, the image information table T4 stores a patient ID, an examination ID, a patient name, an examination date & time, an image type, existence in primary DB, existence in center DB, a storage path in primary DB and a storage path in center DB with respect to the medical facility A.

The GW 47 acquires image data and accessory information (the patient ID, the examination ID, the patient name, the examination date & time, the image type and the like) on the image data from the examination device 5A. That is, the GW 47 functions as an acquirement unit. The accessory information may be inputted from the operation unit 42 or received from the outside by the communication unit 44.

The control unit 41 stores the image data acquired from the examination device 5A in the primary DB 462 of the storage unit 46, and adds a new record to the image information table T4 on the basis of the accessory information (the patient ID, the examination ID, the patient name, the examination date & time, the image type and the like) acquired from the examination device 5A. In addition, with respect to the corresponding examination in the image information table T4, the control unit 41 puts “YES” in the “existence in primary DB”, puts “NO” in the “existence in center DB”, adds a storage location of the image data in the primary DB 462 to the “storage path in center DB”, and leaves the “storage path in center DB” blank.

The control unit 41 controls the communication unit 44 so that the image data stored in the primary DB 462 of the storage unit 46 and the accessory information (the patient ID, the examination ID, the patient name, the examination date & time, the image type, the storage path in primary DB and the like) are transmitted to the data management server 1 so as to be stored in the center DB 162 of the data management server 1. When a notice that the image data is stored in the center DB 162 and a storage location of the image data in the center DB 162 are received by the communication unit 44 from the data management server 1, with respect to the corresponding examination in the image information table T4, the control unit 41 changes the “existence in center DB” to “YES”, and adds the storage location of the image data in the center DB 162 to the “storage path in center DB”.

If a predetermined condition is satisfied, the control unit 41 deletes the image data stored in the primary DB 462 of the storage unit 46 from the primary DB 462. That is, the control unit 41 functions as a delete unit. More specifically, when a predetermined period (for example, one month, three months or the like) elapses from the time the image data is stored in the primary DB 462 of the storage unit 46, the control unit 41 deletes the image data from the primary DB 462. Preferably, as the predetermined period, a period during which image data is normally referred to in the internal system 2A be set. With respect to the examination to which the deleted image data belongs in the image information table T4, the control unit 41 changes the “existence in primary DB” to “NO”, and deletes the storage location of the image data in the primary DB 462 from the “storage path in primary DB”. In addition, the control unit 41 controls the communication unit 44 so that a notice that the image data is deleted from the primary DB 462 is transmitted to the data management server 1.

The control unit 41 searches the primary DB 462 and the center DB 162 for image data, and with respect to image data stored in the primary DB 462, acquires the image data from the primary DB 462, and displays an image on the display unit 43 on the basis of the image data acquired from the primary DB 462, and with respect to image data not stored in the primary DB 462, acquires the image data from the center DB 162, and displays an image on the display unit 43 on the basis of the image data acquired from the center DB 162. That is, the control unit 41 functions as a display control unit.

When an application program 163 newer than the application program 461 already downloaded and stored in the storage unit 46 exists in the data management server 1, the control unit 41 downloads the newer application program 163 from the data management server 1. That is, the control unit 41 functions as a program update unit. More specifically, the control unit 41 compares version information on the already-downloaded application program 461 with the version information on the latest application program 163 existing in the data management server 1, and when the version of the already-downloaded application program 461 is not the latest, controls the communication unit 44 so that an acquirement request for the latest application program 163 is transmitted to the data management server 1 via the communication network N. When the latest application program 163 is received by the communication unit 44 from the data management server 1 via the communication network N, the control unit 41 stores this application program 163 as the application program 461 in the storage unit 46.

The examination device 5A takes an image of a diagnosis target site of a patient, the diagnosis target site being a subject, and performs digital conversion on the taken image so as to generate a medical-use image. Examples of the examination device 5A include CR, CT and MRI.

The internal systems 2B and the like other than the internal system 2A are similar to the internal system 2A, and hence description thereof is omitted. The terminal devices 4B and the like are similar to the terminal 4A, and hence description thereof is omitted by citing FIG. 5.

### [Operations of Data Management System 100]

Next, operations of the data management system 100 are described.

### [Application Start Time Process]

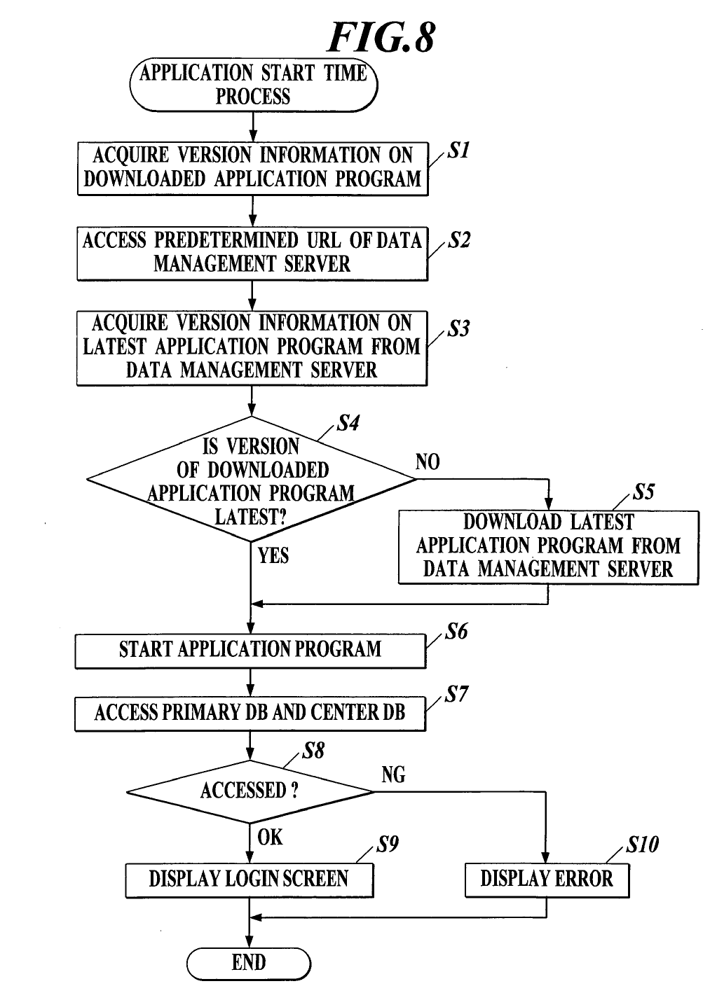

FIG. 8 is a flowchart showing an application start time process performed by the terminal device 4A of the internal system 2A. This process is realized by a software process performed by the control unit 41 in cooperation with the application program 461 stored in the storage unit 46.

First, the control unit 41 acquires the version information on the application program 461 already downloaded and stored in the storage unit 46 (Step S1).

Next, the control unit 41 accesses a predetermined URL (Uniform Resource Locator) of the data management server 1 via the communication network N (Step S2). More specifically, the control unit 41 controls the communication unit 44 so that the predetermined URL is transmitted to the data management server 1.

In the data management server 1, when the communication unit 14 receives the predetermined URL, the control unit 11 reads the version information on the latest application program 163 stored in the storage unit 16, and the communication unit 14 transmits the version information on the latest application program 163 to the terminal device 4A via the communication network N.

In the terminal device 4A, the control unit 41 acquires the version information on the latest application program 163 from the data management server 1 with the communication unit 44 (Step S3).

Then, the control unit 41 determines whether or not the version of the already-downloaded application program 461 is the latest on the basis of the version information on the already-downloaded application program 461 acquired at Step S1 and the version information on the latest application program 163 acquired at Step S3 (Step S4). When determining that the version of the already-downloaded application program 461 is not the latest (Step S4; NO), namely, when determining that an application program 163 newer than the already-downloaded application program 461 exists in the data management server 1, the control unit 41 downloads the latest application program 163 from the data management server 1 (Step S5). More specifically, the control unit 41 controls the communication unit 41 so that an acquirement request for the latest application program 163 is transmitted to the data management server 1 via the communication network N. When the communication unit 44 receives the latest application program 163 from the data management server 1 via the communication network N, the control unit 41 stores this application program 163 as the application program 461 in the storage unit 46.

At Step S4, when determining that the version of the already-downloaded application program 461 is the latest (Step S4; YES), or after Step S5, the control unit 41 starts the application program 461 (Step S6).

Next, the control unit 41 accesses the primary DB 462 of the storage unit 46 and the center DB 162 of the storage unit 16 of the data management server 1 (Step S7). When the access to both the primary DB 462 and the center DB 162 succeeds (Step S8; OK), the control unit 41 displays a login screen on the display unit 43 (Step S9).

On the other hand, when the access to the primary DB 462 or the center DB 162 fails (Step S8; NG), the control unit 41 displays a message that an error occurs on the display unit 43 (Step S10).

Then, the application start time process ends.

### [Image Capture Process]

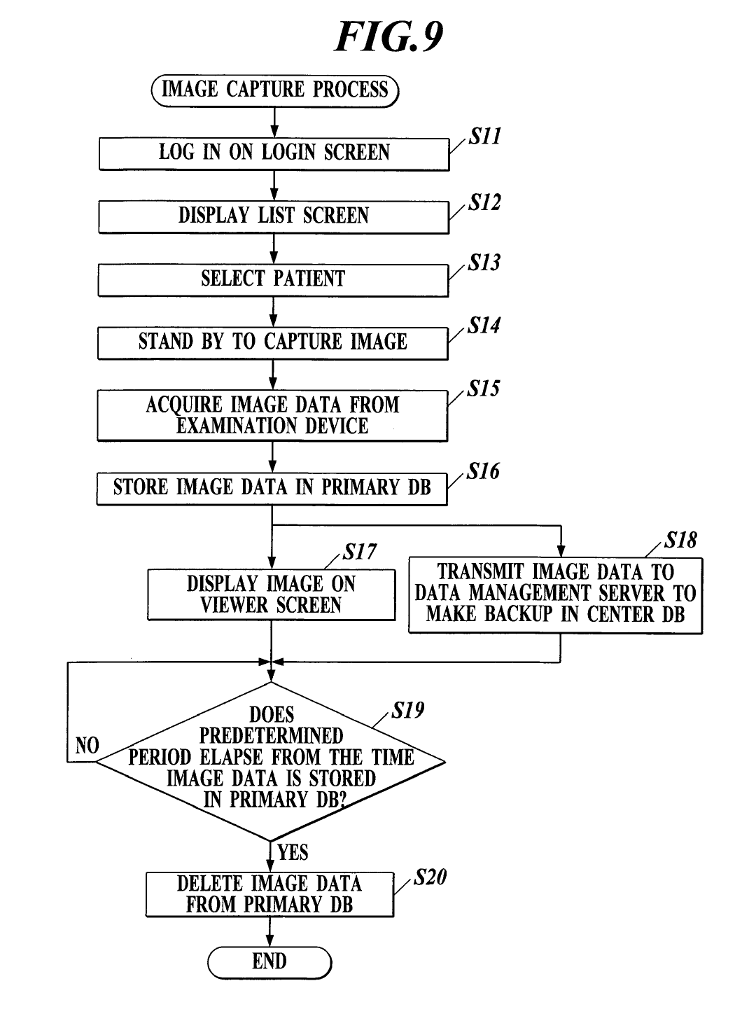

FIG. 9 is a flowchart showing an image capture process performed by the terminal device 4A of the internal system 2A. This process is realized by a software process performed by the control unit 41 in cooperation with the application program 461 stored in the storage unit 46.

First, a user ID and a user PW are inputted to the login screen displayed on the display unit 43 by an operation made with the operation unit 42 (Step S11). The control unit 41 refers to the facility information table T3 contained in the primary DB 462, and permits the use when the inputted user ID and user PW agree with a user ID and a user PW stored in the facility information table T3.

Next, the control unit 41 accesses the primary DB 462 of the storage unit 46 and the center DB 162 of the storage unit 16 of the data management server 1, and displays a list screen on the display unit 43 on the basis of information agreeing with a search condition (Step S12).

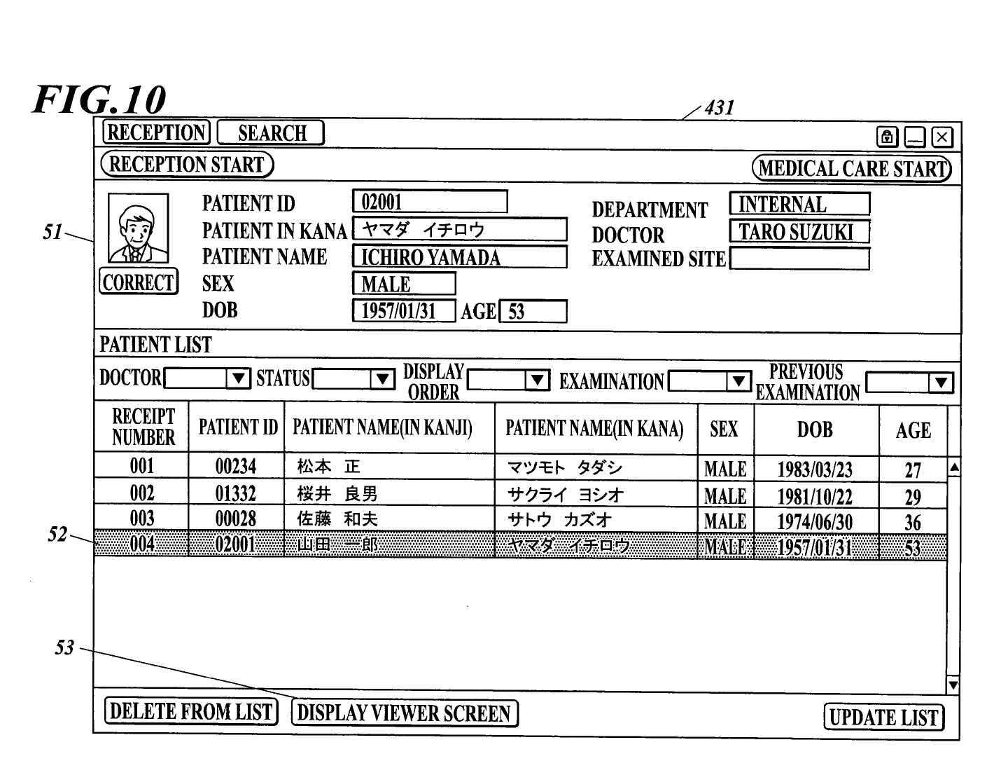

FIG. 10 shows an example of a received patient list screen 431. On the received patient list screen 431, a list of patients having undergone the reception process at the medical facility A on that date is displayed. More specifically, the control unit 41 acquires patient IDs and receipt numbers for patients who have undergone the reception process on that date from the receiving device 3A, and on the basis of the acquired patient IDs, acquires patient information on the patients from the primary DB 462 of the storage unit 46 and the center DB 162 of the storage unit 16 of the data management server 1. Then, the control unit 41 displays the received patient list screen 431 on the display unit 43 on the basis of the acquired patient information. In a patient information display region 51 of the received patient list screen 431, patient information on a patient 52 selected on the list is displayed.

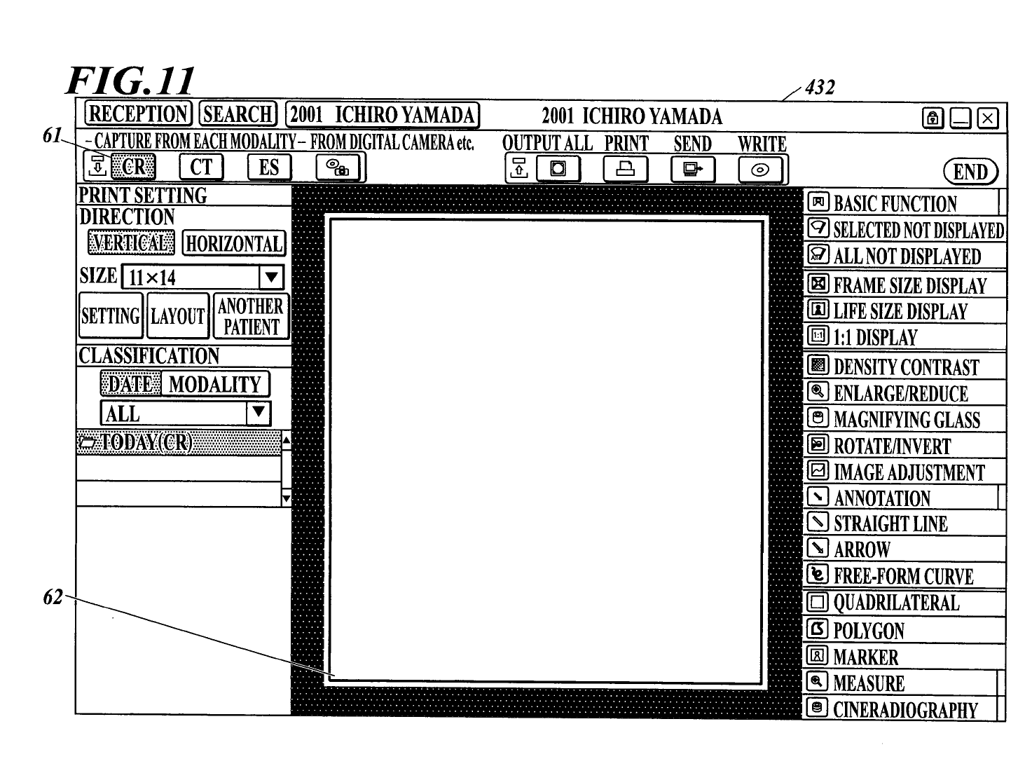

Next, a patient is selected on the list screen displayed on the display unit 43 by an operation made with the operation unit 42 (Step S13). For example, on the received patient list screen 431 shown in FIG. 10, when a patient is selected, and a viewer screen display button 53 is pressed, the control unit 41 displays a viewer screen 432 shown in FIG. 11 on the display unit 43 so as to be in a standby state to capture an image (Step S14).

Next, the GW 47 acquires image data from the examination device 5A (Step S15), and the control unit 41 stores the image data acquired from the examination device 5A in the primary DB 462 of the storage unit 46 (Step S16). In addition, the GW 47 acquires the accessory information (the patient ID, the examination ID, the patient name, the examination date & time, the image type and the like) from the examination device 5A. Then, the control unit 41 stores the accessory information (the patient ID, the examination ID, the patient name, the examination date & time, the image type and the like) in the image information table T4, and in the image information table T4, puts “YES” in the “existence in primary DB”, puts “NO” in the “existence in center DB”, adds a storage location of the image data in the primary DB 462 to the “storage path in primary DB”, and leaves the “storage path in center DB” blank.

Next, the control unit 41 displays an image on the display unit 43 on the basis of the acquired image data (Step S17). For example, when a CR capture button 61 is pressed on the viewer screen 432 shown in FIG. 11 by an operation made with the operation unit 42, the GW 47 acquires the image data and the accessory information from the examination device 5A, and the control unit 41 displays an image in an image display region 62 of the viewer screen 432.

In addition, when the control unit 41 stores the image data in the primary DB 462, the control unit 41 controls the communication unit 44 so that the image data is transmitted to the data management server 1, whereby a backup of the image data is made in the center DB 162 of the storage unit 16 of the data management server 1 (Step S18). At the time, the accessory information (the patient ID, the examination ID, the patient name, the examination date & time, the image type, the storage path in primary DB and the like) is also transmitted to the data management server 1. Step S18 is realized by always (for example, at intervals of three seconds) comparing the image data stored in the primary DB 462 with the image data stored in the center DB 162, and executing a resident program to start copying data when detecting that new image data is stored in the primary DB 264. When the communication unit 44 receives a notice that the image data is stored in the center DB 162 and a storage location of the image data in the center DB 162 from the data management server 1, with respect to the corresponding examination in the image information table 4, the control unit 41 changes the “existence in center DB” to “YES”, and adds the storage location of the image data in the center DB 162 to the “storage path in center DB”.

After Step S17 or Step S18, the control unit 41 determines whether or not a predetermined period elapses from the time the image data is stored in the primary DB 462 (Step S19). When determining that a predetermined period elapses from the time the image data is stored in the primary DB 462 (Step S19; YES), the control unit 41 deletes the image data from the primary DB 462 (Step S20). Then, with respect to the corresponding examination in the image information table 4, the control unit 41 changes the “existence in primary DB” to “NO”, and deletes the storage location of the image data in the primary DB 462 from the “storage path in primary DB”. In addition, the control unit 41 controls the communication unit 44 so that a notice that the image data is deleted from the primary DB 462 is transmitted to the data management server 1.

Then, the image capture process ends.

### [Image Display Process]

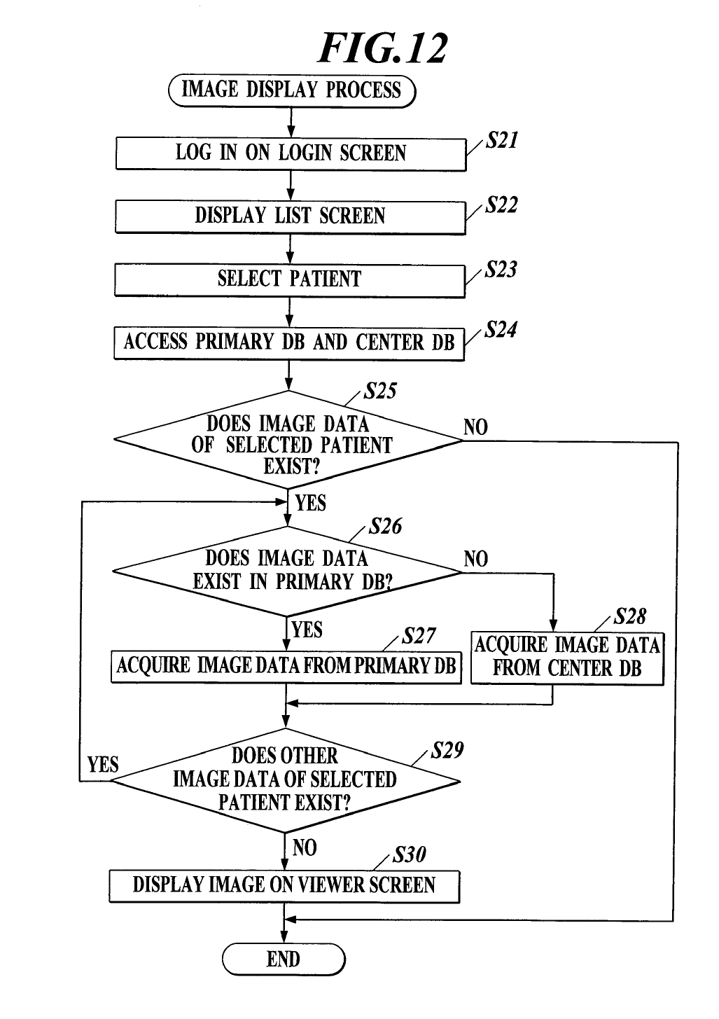

FIG. 12 is a flowchart showing an image display process performed by the terminal device 4A of the internal system 2A. This process is realized by a software process performed by the control unit 41 in cooperation with the application program 461 stored in the storage unit 46.

First, a user ID and a user PW are inputted to the login screen displayed on the display unit 43 by an operation made with the operation unit 42 so as to log in (Step S21).

Next, the control unit 41 accesses the primary DB 462 of the storage unit 46 and the center DB 162 of the storage unit 16 of the data management server 1, and displays a list screen on the display unit 43 on the basis of information agreeing with a search condition (Step S22).

Next, a patient is selected on the list screen displayed on the display unit 43 by an operation made with the operation unit 42 (Step S23).

Next, the control unit 41 accesses the primary DB 462 of the storage unit 46 and the center DB 162 of the storage unit 16 of the data management server 1 (Step S24). When image data of the selected patient exists in the primary DB 462 or the center DB 162 (Step S25; YES), the control unit 41 determines whether or not the image data of the selected patient exists in the primary DB 462 (Step S26). More specifically, the control unit 41 refers to the column “existence in primary DB” in the image information table T4 contained in the primary DB 462 with respect to each examination performed on the selected patient, and in the case of “YES”, determines that the image data exists in the primary DB 462, and in the case of “NO”, determines that the image data does not exist in the primary DB 462.

When determining that the image data of the selected patient exists in the primary DB 462 (Step S26; YES), the control unit 41 refers to the column “storage path in primary DB” in the image information table T4 with respect to the corresponding examination so as to acquire the image data from the primary DB 462 (Step S27).

On the other hand, when determining that the image data of the selected patient does not exist in the primary DB 462 (Step S26; NO), the control unit 41 refers to the column “storage path in center DB” in the image information table T4 with respect to the corresponding examination so as to acquire the image data from the center DB 162 (Step S28). More specifically, the control unit 41 controls the communication unit 44 so that an acquirement request for the image data specified by the “storage path in center DB” is transmitted to the data management server 1 via the communication network N.

In the data management server 1, the control unit 11 reads the image data specified by and corresponding to the “storage path in center DB” from the center DB 162, and the communication unit 14 transmits the read image data to the terminal device 4A.

In the terminal device 4A, the communication unit 44 receives the image data transmitted from the data management server 1.

After Step S27 or Step S28, when other image data of the selected patient exists (Step S29; YES), the control unit 41 returns to Step S26, and repeats the process for the other image data. At Step S29, when other image data of the selected patient does not exists (Step S29; NO), the control unit 41 displays an image or images on the display unit 43 on the basis of the acquired one or more image data (Step S30).

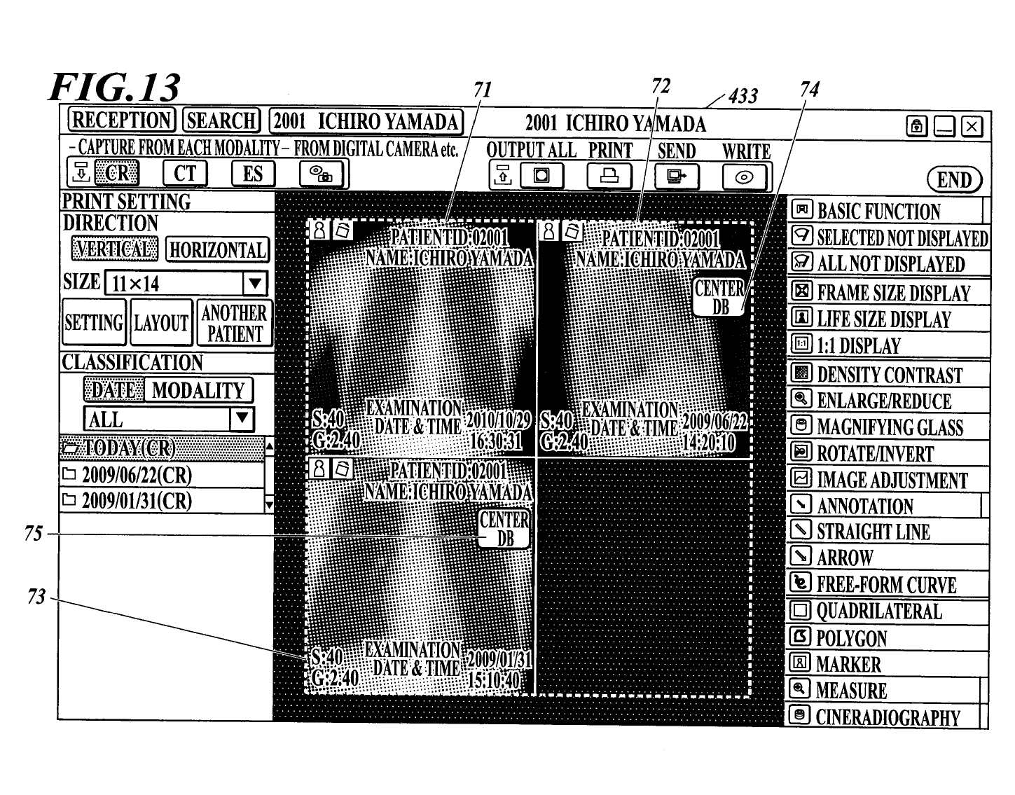

FIG. 13 shows an example of a viewer screen 433. An image based on the image data acquired from the primary DB 462 is displayed in an image display region 71. Images based on the image data acquired from the center DB 162 are displayed in image display regions 72 and 73. Center DB marks 74 and 75 are displayed for the images based on the image data acquired from the center DB 162 so as to be distinguishable from the image based on the image data acquired from the primary DB 462.

In the data management server 1, data compression, resolution thinning or the like may be performed on image data in advance so as to generate a compressed image, and when the image data is acquired from the center DB 162 of the terminal device 4A, the compressed image may be acquired and displayed first.

At Step S25, when image data of the selected patient does not exist in either the primary DB 462 or the center DB 162 (Step S25; NO), the process ends there.

Then, the image display process ends.

### [Fee Calculation Process]

FIG. 14 is a flowchart showing a fee calculation process performed by the data management server 1. This process is performed for each medical facility, and realized by a software process performed by the control unit 11 in cooperation with the server program 161 stored in the storage unit 16.

First, the control unit 11 determines whether or not now is the timing to calculate a fee (Step S31). For example, a certain date of each month is predetermined as a fee calculation day.

When determining that now is the timing to calculate a fee (Step S31; YES), the control unit 11 acquires the number of software used times, the data storage amount and the number of cooperative service used times with respect to a target medical facility from the facility information table T1 contained in the center DB 162 (Step S32). Then, the control unit 11 calculates a fee for use of the data management system 100 by the target medical facility (terminal device 4A, 4B or . . . ) on the basis of the acquired number of software used times, data storage amount and number of cooperative service used times (Step S33). For example, the fee increases as the number of software used times increases, the fee increases as the data storage amount increases, and the fee increases as the number of cooperative service used times increases. However, a calculation method of the fee for use thereof is not limited thereto.

Then, the fee calculation process ends.

As described above, according to the embodiment, in the terminal device 4A, 4B and . . . , the image data stored in the primary DB 462 is acquired from the primary DB 462, and the image data not stored in the primary DB 462 (the image data deleted from the primary DB 462 as a predetermined period elapses) is acquired from the center DB 162. Accordingly, time necessary for displaying images can be reduced, and the diagnostic efficiency can be prevented from decreasing. Usually, image data generated by an examination is referred to right after the examination. Hence, by keeping the image data in the primary DB 462 for a predetermined period, images can be displayed promptly even in a not-high-speed communication environment.

Furthermore, when an application program 163 newer than the already-downloaded application program 461 exists in the data management server 1, the terminal device 4A, 4B and . . . automatically downloads the newer application program 163 from the data management server 1. Accordingly, the latest application program can be used always, and also user's trouble can be reduced.

Furthermore, in the data management server 1, fees can be calculated in accordance with the use of the data management system 100 by the terminal device 4A, 4B and . . . .

The above-described embodiment is an example of the data management system of the present invention, and hence the present invention is not limited thereto. In addition, the detailed configurations and detailed operations of the devices constituting the system can be appropriately modified without departing from the spirit of the present invention.

For example, in the embodiment, in the terminal device 4A, 4B and . . . , image data is deleted from the primary DB 462 when a predetermined period elapses from the time the image data is stored in the primary DB 462. However, image data may be deleted in order of a period stored in the primary DB 462 with the longest one first when the data amount of data stored in the storage unit 46 reaches the storage capacity of the storage unit 46 (or when the data amount in the primary DE 462 reaches a predetermined storage capacity usable as the primary DB 462).

In the above, as a computer readable medium to store the programs to perform the processes, an HDD or a nonvolatile memory is used. However, this is not a limitation. As another computer readable medium, a portable storage medium such as a CD-ROM can be used. Furthermore, as a medium to provide data of the programs via a communication line, a carrier wave can be used.

### INDUSTRIAL APPLICABILITY

The information processing device and the data management system of the present invention have applicability in the medical field in which image data acquired from examination devices is managed.

### DESCRIPTION OF THE NUMERALS

1 Data Management Server
2A, 2B Internal System
3A, 3B Receiving Device
4A, 4B Terminal Device
5A, 5B Examination Device
6A, 6B Router
7A, 7B Internal Network
11 Control Unit
12 Operation Unit
13 Display Unit
14 Communication Unit
15 RAM
16 Storage Unit
17 Bus
41 Control Unit
42 Operation Unit
43 Display Unit
44 Communication Unit
45 RAM
46 Storage Unit
47 GW
48 Bus
100 Data Management System
161 Server Program
162 Center DB
163 Application Program
461 Application Program
462 Primary DB
N Communication Network

1. An information processing device comprising:
an acquirement unit which acquires image data from an examination device;
a first storage unit which temporarily stores the image data acquired from the examination device;
a communication unit which transmits the image data stored in the first storage unit to a data management server so as to store the image data in a second storage unit of the data management server connected to the communication unit via a communication network so as to perform data communication;
a delete unit which deletes the image data stored in the first storage unit from the first storage unit in response to a predetermined condition being satisfied; and
a display control unit which searches the first storage unit and the second storage unit for the image data, acquires the image data from the first storage unit and displays an image on a display unit on the basis of the image data acquired from the first storage unit with respect to the image data stored in the first storage unit, and acquires the image data from the second storage unit and displays an image on the display unit on the basis of the image data acquired from the second storage unit with respect to the image data not stored in the first storage unit.

2. The information processing device according to claim 1, wherein the delete unit deletes the image data stored in the first storage unit from the first storage unit in response to a predetermined period elapsing from when the image data is stored in the first storage unit.

3. The information processing device according to claim 1, wherein
a process performed by the information processing device is performed in accordance with an application program downloaded from the data management server, and
the information processing device further comprises a program update unit which, in response to a newer application program than the downloaded application program existing in the data management server, downloads the newer application program from the data management server.

4. A data management system comprising:
an information processing device including a first storage unit which temporarily stores image data acquired from an examination device; and
a data management server including a second storage unit which stores the image data transmitted from the information processing device connected to the data management server via a communication network so as to perform data communication, wherein
the information processing device includes:
a communication unit which transmits the image data stored in the first storage unit to the data management server so as to store the image data in the second storage unit;
a delete unit which deletes the image data stored in the first storage unit from the first storage unit in response to a predetermined condition being satisfied; and
a display control unit which searches the first storage unit and the second storage unit for the image data, acquires the image data from the first storage unit and displays an image on a display unit on the basis of the image data stored in the first storage unit with respect to the image data stored in the first storage unit, and acquires the image data from the second storage unit and displays an image on the display unit on the basis of the image data stored in the second storage unit with respect to the image data not stored in the first storage unit.

5. The data management system according to claim 4, wherein the delete unit deletes the image data stored in the first storage unit from the first storage unit in response to a predetermined period elapsing from when the image data is stored in the first storage unit.

6. The data management system according to claim 4, wherein
a process performed by the information processing device is performed in accordance with an application program downloaded from the data management server, and
the information processing device further includes a program update unit which, in response to a newer application program than the downloaded application program existing in the data management server, downloads the newer application program from the data management server.

7. The data management system according to claim 6, wherein the data management server further includes a fee calculation unit which calculates a fee for use of the data management system by the information processing device on the basis of a data amount of the image data stored in the second storage unit and a number of times the application program is used.
